SEQUENCE LISTING (16 sequences)

SEQ ID NO: 1 - RNA, 21 nt, Artificial Sequence
  Silencing RNA sequence; SMURF-1 sense strand
  misc_feature (20)..(21): n=dt
gagauaugagagggacuuann

SEQ ID NO: 2 - RNA, 21 nt, Artificial Sequence
  Silencing RNA sequence; SMURF-1 anti-sense strand
  misc_feature (20)..(21): n=dt
  misc_feature (21)..(21): y=dg
uaagucccuaucauaucucny

SEQ ID NO: 3 - RNA, 21 nt, Artificial Sequence
  Silencing RNA sequence; SMURF-2 sense strand
  misc_feature (20)..(21): n=dt
ggcuucaccacaucaugaann

SEQ ID NO: 4 - RNA, 21 nt, Artificial Sequence
  Silencing RNA sequence; SMURF-2 anti-sense strand
  misc_feature (20)..(20)
  misc_feature (21)..(21): n=dt
uucaugauguggugaagccyn

SEQ ID NO: 5 - RNA, 21 nt, Artificial Sequence
  Silencing RNA sequence; SMURF-3 sense strand
  misc_feature (20)..(21): n=dt
gcguuuggaucuaugcaaann

SEQ ID NO: 6 - RNA, 21 nt, Artificial Sequence
  Silencing RNA sequence; SMURF-3 anti-sense strand
  misc_feature (20)..(20): n=dt
  misc_feature (21)..(21): y=dg
uuugcauagauccaaacgcny

SEQ ID NO: 7 - RNA, 21 nt, Artificial Sequence
  Silencing RNA sequence; SMURF-4 sense strand
  misc_feature (20)..(21): n=dt
cauuuauucuccuuuauuann

SEQ ID NO: 8 - RNA, 19 nt, Artificial Sequence
  Silencing RNA sequence; SMURF-4 anti-sense strand
  misc_feature (20)..(21): y=dg
auaaaggagaauaaaugyy

SEQ ID NO: 9 - RNA, 21 nt, Artificial Sequence
  Silencing RNA sequence; HDAC-1 sense strand
  misc_feature (20)..(21): n=dt
agaagaugaucgucuucaann

SEQ ID NO: 10 - RNA, 21 nt, Artificial Sequence
  Silencing RNA sequence; HDAC-1 anti-sense strand
  misc_feature (20)..(20): n=dt
  misc_feature (21)..(21): b=da
uugaagacgaucaucuucunb

SEQ ID NO: 11 - RNA, 21 nt, Artificial Sequence
  Silencing RNA sequence; HDAC-2 sense strand
  misc_feature (20)..(21): n=dt
cggugcuggacauaugaaann

SEQ ID NO: 12 - RNA, 21 nt, Artificial Sequence
  Silencing RNA sequence; HDAC-2 anti-sense strand
  misc_feature (20)..(21): y=dg
uuucauauguccagcaccgyy

SEQ ID NO: 13 - RNA, 21 nt, Artificial Sequence
  Silencing RNA sequence; HDAC-3 sense strand
  misc_feature (20)..(21): n=dt
gagacuguuagagaugaaann

SEQ ID NO: 14 - RNA, 21 nt, Artificial Sequence
  Silencing RNA sequence; HDAC-3 anti-sense strand
  misc_feature (20)..(20): n=dt
  misc_feature (21)..(21): y=dg
uuucaucucuaacagucucny

SEQ ID NO: 15 - RNA, 21 nt, Artificial Sequence
  Silencing RNA sequence; HDAC-4 sense strand
  misc_feature (20)..(21): n=dt
caaugaauucuaugauggann

SEQ ID NO: 16 - RNA, 21 nt, Artificial Sequence
  Silencing RNA sequence; HDAC-4 anti-sense strand
  misc_feature (21)..(21): y=dg
uccaucauagaauucauugyy

(19) United States
(12) Patent Application Publication
(10) Pub. No.: US 2008/0038308 A1
(43) Pub. Date: Feb. 14, 2008

(54) Silencing RNA molecules and their use in bone formation

(75) Inventors: Stephen George Miller, High Springs, FL (US); Peter R. Supronowicz, Gainesville, FL (US); Jordan Michael Katz, Gainesville, FL (US)

Correspondence Address: MCANDREWS HELD & MALLOY, LTD, 500 WEST MADISON STREET, SUITE 3400, CHICAGO, IL, 60661, US

(73) Assignee: Regeneration Technologies, Inc.

(21) Appl. No.: 11/496,954

(22) Filed: Aug. 1, 2006

Related U.S. Application Data
(60) Provisional application No. 60/704,484, filed on Aug. 1, 2005.

(51) Int. Cl. A61K 31/711; A61K 9/00; A61P 19/00; C07H 21/02

(52) U.S. Cl. 424/423; 514/44; 536/24.5

(57) ABSTRACT

The present invention is directed to siRNA molecules that down-regulate the expression of proteins that inhibit bone formation. In another aspect, the instant invention is directed to compositions and/or implants comprising in combination such siRNA molecules in a pharmaceutically acceptable carrier or implant.

This application claims the benefit of provisional U.S. Patent Application Ser. No. 60/704,484, filed Aug. 1, 2005, the whole of which is incorporated herein by reference.

### FIELD OF THE INVENTION

The present invention is related to the field of silencing RNA (siRNA), specifically to removing inhibitors of tissue growth, healing or regeneration, including new bone formation, by modulation of expression and/or activity of genes with silencing RNA. More specifically, the present invention is directed to siRNA molecules and delivery systems which down-regulate osteo-inhibitory gene products and/or enhance bone-forming gene expression by reduction of levels of inhibitory proteins. The siRNA molecules and composites of the present invention are useful as pharmaceutical agents in mammalian patients, particularly human patients, because they offer improved clinical outcome of the repair of bony defects, especially in the at-risk (osteoporotic, diabetic, smoker, etc.) patient population.

### BACKGROUND OF THE INVENTION

In the field of medicine, there has been an increasing need to develop implant materials that accelerate the rate of bone formation in patients in need of repair of bone defects. As the body ages, the number of bone marrow stem cells decline and healing rates decline. Similarly, bone regeneration represents a clinical challenge in cases such as severe trauma, femoral necrosis, restoration following surgical ablation of tumors, and induction of spinal fusion. Clinical intervention in the cascade of wound healing events requires an understanding of the complex interaction of cells, cytokines, bone matrix and remodeling enzymes.

A comprehensive picture of the bone morphogenic protein (BMP) signaling pathway at the molecular level is key to achieving bone regeneration. (BMPs) are multifunctional regulators of cell growth, differentiation and apoptosis that belong to the transforming growth factor (TGF)-.beta. superfamily. More than a dozen members of the BMP protein family have been identified in mammals, which can be subclassified into several groups depending on their structures. BMP-2 and BMP-4 are highly similar to each other. BMP-5, BMP-6, osteogenic protein (OP)-1 (also called BMP-7), and OP-2/BMP-8 are structurally similar to each other. Growth-differentiation factor (GDF)-5 (also termed cartilage-derived morphogenetic protein-1), GDF-6 (also cartilage-derived morphogenetic protein-2), and GDF-7 form another related group. In contrast to BMP-2, BMP-4, BMP-6, and OP-1/BMP-7, which induce bone and cartilage formation in vivo, GDF-5, GDF-6, and GDF-7 more efficiently induce cartilage and tendon-like structures in vivo.

Dimeric BMP proteins (either embedded in bone matrix or secreted from osteoblasts at the fracture site) initiate signaling at the cell surface by interacting with two serine/threonine kinase receptors that when occupied are able to activate intracellular SMAD signaling proteins via phosphorylation. Phosphorylated receptor-regulated SMADS (SMADS 1, 5 and 8) are then able to form a complex with SMAD 4 which translocates to the nucleus. This complex, which itself can take on additional positively- or negatively-acting proteins, binds to other regulatory proteins associated with the promoters of genes conferring osteoblastic phenotypes (Groeneveld and Berger, 2000; Canalis et al., 2003). The principle co-transcriptional activator protein in cells determined to become osteoblasts is runx2, a protein that serves as a molecular scaffold to which other transcriptional factors bind (Ducy et al., 1997; Yang et al., 2003; Stein et al., 2004). Binding of the SMAD transcriptional complex is a prerequisite for runx2 to become a fully-functional positive transcriptional regulator. Attenuation of runx2 function accompanying the cessation of bone formation is achieved through either the binding of negative regulatory molecules to the transcriptional scaffold or by autoregulation of runx2 transcription (Geoffroy et al., 2002; Canalis et al., 2003). Various post-translational modifications of runx2 including phosphorylation also serve to regulate patterns of transcription in osteoblasts (Fujita et al., 2001).

Likewise, an understanding of the types of cells responsive to BMPs and that mediate bone formation is also important (Einhom et al., 1998; Gerstenfeld et al., 2003). Morphogenetic fields of bone repair are defined by relationships between damaged calcified tissue with surrounding soft tissues. Potential sources of repair cells and signals that define these fields include the periosteum, the adjacent soft tissues, and the marrow space at the site of damaged bone. While the periosteum contributes fully-differentiated osteoblasts for bone formation, pluripotent mesenchymal stem cells in the marrow require a further inductive step mediated by BMPs, while surrounding differentiated tissue (such as muscle, which itself is of mesenchymal origin) requires yet an additional de-differentiative step that is also BMP-dependent (Iwata et al., 2002; Jingushi et al., 2002). Marrow stromal cells are capable of undergoing differentiation not only into osteoblasts but also into chondrocytes, adipocytes, fibroblasts and myoblasts given appropriate stimuli (Bianco et al., 2001; Sekiya et al., 2002). Demineralized bone matrix (DBM) is capable of reproducing these BMP-mediated effects either in vitro or in animal models although with a lower efficiency than is seen with purified BMPs used at supra-physiologic levels.

It is estimated that over 35 million individuals per year sustain musculoskeletal injuries that could theoretically be treated with some type of bone grafting procedure (synthetic, autograft, allograft or xenograft). For example, the annual number of non-union fractures is estimated at almost 4 million globally. Bone grafts with improved properties could also be used in procedures that do not currently use bone grafting such as arthroplasty, artificial hips, and artificial knees. Nearly 10 million Americans suffer from osteoporosis which by itself is responsible for nearly 1.5 million fractures every year. The fractures that occur in older patients (over 65), in diabetics, or in persons who smoke present especially challenging environments for healing due to the reduced number of both osteoblasts and stem cells available. Most of these patients would therefore benefit from an improved graft product having an increased healing rate.

The instant invention provides a safer, more cost-effective alternative in areas where patients and their care providers currently have relatively few options. The area currently served by BMP-based products is one such example. Not only are indications currently somewhat limited for cytokine-delivery devices (restricted to single level lumbar fusions in the case of InFuse® (Medtronic Sofamor Danek), for example) but a 2 mg-5 mg dosage costs in the thousands of dollars including sponge and cage. By contrast a bone paste delivery system containing siRNA would be less costly, while also taking advantage of the osteoinductive and osteoconductive properties of DBM to enhance the amount of induced bone growth.

Demineralized bone matrix (DBM)-based paste products currently used as general orthopedic grafts show useful bone repair properties when used at anatomic sites that experience some loading such as hip revisions, long bone fractures, and spinal fusions. These clinical findings are consistent with the osteoinductive (i.e., osteoblast-inducing) and osteoconductive (cell binding-permissive) nature of these natural bone-derived materials (Zhang et al. 1997; Chesmel et al., 1998; Hartman et al., 2004). Nonetheless, DBM-based products give less than satisfactory results in non-load bearing situations and in more challenging posterolateral spinal fusions (Nuschik et al., 2000; Sandhu et al., 2001).

At the level of gene transcription, the initiation of new bone formation is now understood to be a result of the up-regulation of positive transcriptional regulators and their induced products and the down-regulation of inhibitors of transcriptional activation that interfere with the BMP signaling cascade. Interestingly, when even a single signaling inhibitor is removed, enhanced bone formation is both quantifiable by biochemical criteria and is clinically-significant (see below and Canalis et al., 2003; Lee et al., 2003 for recent reviews).

siRNA is a technology that exploits the observation that specific RNAs are targeted for degradation by short, complementary double stranded RNAs. This phenomenon, which appears to be common to all metazoa, is mediated by a conserved adaptation known as “RNA interference.” These events are carried out by a host of proteins that form functional aggregates in the cytoplasm including: a member of the RNAse III family of nucleases called “dicer” which cleaves long double stranded RNAs into smaller 21-23 bp duplexes; and the RISC (RNA-induced silencing complex) complex comprised of proteins that bind the RNA duplex, unwind the duplex to expose a single strand for Watson-Crick base pairing with a target, and a nuclease that causes nucleolytic cleavage of the target RNA (McManus and Sharp, 2002; Bantounas et al., 2004).

These events, which represent an adaptation to prevent viral replication and transposable element mobilization, can be induced artificially. A significant breakthrough in this area occurred with a report by Elbashir and coworkers (2001) that siRNA could be induced in cultured mammalian cells using chemically-synthesized 21-nucleotide siRNA molecules. This finding was important since introduction of long double stranded RNAs into cells triggered a “panic response,” a non-specific inhibition of all cellular transcription and the induction of interferon-alpha. In addition to the introduction of dsRNAs into cells by transfection, siRNAs can be delivered via transfection of cells with plasmids encoding “hairpin” RNAs under the control of U6 promoters (Gou et al., 2003; Silva et al., 2004) or by using retroviral vector delivery/expression systems (Ichim et al., 2004). These methods provide tools for determining the function of newly-characterized genes through the removal of their encoded RNA and protein phenotype. It is an object of the present invention to discover siRNA molecules that down-regulate specific proteins that inhibit osteogenesis. It is a further object of this invention to use these siRNA molecules as active agents in pharmaceutical compositions and implants for providing positive clinical outcomes in cases where over expression or inappropriate expression of a particular gene leads to a disease state.

In view of the foregoing considerations, there has been a long felt need for implant materials that accelerate the rate of healing, e.g., bone formation, in patients in need of the repair of bone or other defects. It is a further object of this invention to provide various siRNA molecules and delivery systems (implant materials) which down-regulate osteo-inhibitory gene products and/or enhance bone-forming gene expression by the reduction of inhibitory proteins. It is an additional object of the invention to provide various siRNA molecules and delivery systems which reduce inflammatory proteins at non-bony sites (such as the nucleus pulposus).

### BRIEF SUMMARY OF THE INVENTION

This invention relates to silencing RNA (siRNA)-mediated gene inactivation and it has multiple aspects. In a first aspect, the instant invention is directed to siRNA molecules that down-regulate the expression of proteins that inhibit bone formation. In another aspect, the instant invention is directed to pharmaceutical compositions and/or implants comprising such siRNA molecules in a pharmaceutically acceptable carrier. In many instances, a pharmaceutically acceptable carrier is the implant itself, such as autograft bone, allograft bone, xenograft bone, demineralized bone matrix, atelocollagen, gelatin or another carrier as described in greater detail herein. In its third aspect, the present invention is directed to a method for enhancing osteogenesis or chrondrogenesis in a mammalian patient comprising administering to a patient in need of said osteogenesis or chondrogenesis a siRNA molecule that down-regulates the expression of a protein that is an inhibitor of osteogenesis or chondrogenesis, respectively. The siRNA molecule of the instant invention, when delivered to a wound/defect site and is taken up by non-bone-forming cells, removes an inhibitory barrier to the expression of bone-forming genes and thereby induces an osteoblastic phenotype. The siRNA molecules of the instant invention may be used alone or in combination with one another. The siRNA-containing implants of the instant invention result in better clinical outcomes, rapid return to the labor pool and significant health care cost advantages.

There are several embodiments of the first aspect of the present invention. One embodiment is directed to an siRNA molecule comprising a double stranded RNA portion having about 5 to 40 bp, more typically 13 to 30 bp, most typically 19 to 23 bp, the siRNA molecule down-regulating the expression in a mammal, preferably a human, of a protein inhibitor of osteogenesis. In addition to the double stranded RNA, the siRNA molecules further comprise two single stranded DNA portions, which may be the same or different, each DNA portion comprising two deoxynucleotides (dA, dC, dG and dT), which may be the same or different, attached to the 3′ end of each RNA strand. Typical deoxynucleotide pairs include dTdT, dGdG, dAdA, dTdA, dAdT, dGdT, dTdG, dGdA, dCdA, dCdC, dAdG and dAdC.

In another embodiment, the present invention is directed to an siRNA molecule comprising a double stranded RNA having about 5 to 40 bp, more typically 13 to 30 bp, most typically 19 to 23 bp, the siRNA molecule down-regulating the expression in a mammal, preferably a human, of a protein inhibitor of chondrogenesis. In a preferred embodiment, the chondrogenesis comprises regeneration of a nucleus pulposa in a mammalian patient, typically a human.

The silencing RNA (siRNA) molecules of the present invention are readily synthesized in the laboratory using conventional techniques. They are short double stranded RNAs (about 5 to 40 bp, more typically 13 to 30 bp, and most typically 19 to 23 bp), preferably having deoxynucleotide tails, which may be the same or different, on the respective 3′ ends of their sense and anti-sense strands. These siRNA molecules are water-soluble and when delivered directly to cells either in vitro or in vivo, affect the degradation of the targeted mRNAs (e.g., the mRNAs encoding inhibitory polypeptides) in the cytoplasm. By the practice of the instant invention, cells that are attracted to sites of fracture, sites of necrosis, or sites in which osteoconductive graft materials have been implanted are transformed into bone-forming osteoblasts. By increasing the number of osteoblasts, the rates of matrix formation, matrix mineralization, and new woven bone formation are accelerated. We have identified genes regulating both embryonal skeletonogenesis and healing of bony defects post-partum. Of particular interest are the genes whose products inhibit bone formation in space and time to prevent inappropriate formation of mineralized tissue. Many of these genes, when functionally-inactivated (by naturally-occurring loss of function alleles or in knock-out animals), result in biochemically-quantifiable, clinically-relevant enhancement of bone formation. Applicants have identified siRNA molecules of SEQ ID NOS: 1-16, which when provided in double stranded form to a target mammalian cell, typically a human cell, causes the down-regulation in that cell of the expression one or more proteins that inhibit bone formation (osteogenesis). See FIGS. 1-3. Typical target cells are osteoblasts, pre-osteoblasts and stem cells. Because these target cells are present at the site of any recent injury or surgery to bone, these cells are inherently targeted by merely targeting the injury site.

In one embodiment, the invention is directed to a composition comprising a synthetic, autograft, allograft or xenograft implant material combined with a silencing RNA (siRNA) of the present invention. Examples of two target protein inhibitors are SMURF 1 and HDAC 3. Listed below are 4 different siRNA molecules (designated as −1 to −4) and comprising a pair of complementary (sense and anti-sense) RNA sequences for down regulating their respective protein inhibitors (SMURF 1 or HDAC 3):

| | |
|---|---|
| SMURF-1: | |
| 5′ GAGAUAUGAGAGGGACUUAdTdT 3′ | SEQ ID NO: 1 |
| 3′ dGdTCUCUAUACUAUCCCUGAAU 5′ | SEQ ID NO: 2 |
| SMURF-2: | |
| 5′ GGCUUCACCACAUCAUGAA dTdT 3′ | SEQ ID NO: 3 |
| 3′ dTdGCCGAAGUGGUGUAGUACUU 5′ | SEQ ID NO: 4 |
| SMURF-3: | |
| 5′ GCGUUUGGAUCUAUGCAAAdTdT 3′ | SEQ ID NO: 5 |
| 3′ dGdTCGCAAACCUAGAUACGUUU 5′ | SEQ ID NO: 6 |
| SMURF-4: | |
| 5′ CAUUUAUUCUCCUUUAUUAdTdT 3′ | SEQ ID NO: 7 |
| 3′ dGdGGUAAAUAAGAGGAAAUA 5′ | SEQ ID NO: 8 |
| HDAC-1: | |
| 5′ AGAAGAUGAUCGUCUUCAAdTdT 3′ | SEQ ID NO: 9 |
| 3′ dAdTUCUUCUACUAGCAGAAGUU 5′ | SEQ ID NO: 10 |
| HDAC-2: | |
| 5′ CGGUGCUGGACAUAUGAAAdTdT 3′ | SEQ ID NO: 11 |
| 3′ dGdGGCCACGACCUGUAUACUUU 5′ | SEQ ID NO: 12 |
| HDAC-3: | |
| 5′ GAGACUGUUAGAGAUGAAAdTdT 3′ | SEQ ID NO: 13 |
| 3′ dGdTCUCUGACAAUCUCUACUUU 5′ | SEQ ID NO: 14 |
| HDAC-4: | |
| 5′ CAAUGAAUUCUAUGAUGGAdTdT 3′ | SEQ ID NO: 15 |
| 3′ dGdGGUUACUUAAGAUACUACCU 5′ | SEQ ID NO: 16 |

Alternatively, an siRNA molecule of the present invention comprises any one of the above RNA sequences in combination with a complementary sequence. Thus, while the RNA portions of the molecule would not change, the DNA tail at the 3′ end of the complementary strand would change.

Implant materials include, but are not limited to, moldable materials such as paste; and load-bearing materials, such as machined cortical or cortical-cancellous bone implants of autograft, allograft or xenograft origin. When the implant material (or carrier) is allograft or xenograft bone, the bone is cleansed and sterilized to be antigen- and pathogen-free. A preferred implant material is bone paste made from allograft or xenograft bone. Bone paste products consist of demineralized bone matrix (DBM) combined with a carrier (to enhance viscosity and handling) which is then hydrated in a syringe or other suitable applicator. In one embodiment, commercially available bone paste products such as OSTEOFIL® (Regeneration Technologies, Inc.) are utilized in combination with siRNA molecules. In another embodiment, a specially formulated bone paste product is utilized in combination with siRNA molecules. In yet another embodiment, siRNAs are encapsulated or chemically modified to enhance performance when utilized in combination with a commercially available or specially formulated paste.

DBM itself contains a broad spectrum of cytokines and other osteoinductive factors laid down in bone matrix during skeletonogenesis and subsequent bone remodeling during the lifetime of the donor (Urist). These osteoinductive molecules are made available to uncommitted or partially-differentiated osteoblasts (“bone forming cells”) through the action of matrix metalloproteases which initiate remodeling of bone at fracture sites. These osteogenic materials, which include but are not limited to, bone morphogenetic proteins (BMPs), transforming growth factors (TGFs), fibroblastic growth factors (FGFs), and platelet-derived growth factors (PDGFs), are largely responsible for the induction of new bone growth by DBM grafts. At the molecular level the BMPs released from DBM trigger BMP signaling pathways which give rise to the underlying steps in bone formation attributed to the specialized molecular phenotype of osteoblasts. DBM introduced into an intramuscular pouch in rats recapitulates normal endochondral bone formation (including cartilage (chondrogenesis), bone (osteogenesis), and marrow development) and is a model used to validate the osteoinductivity of DBM-based products.

In another aspect, the instant invention is directed to an implantable composition comprising in combination a demineralized bone matrix (DBM) and an siRNA effector molecule of the present invention. In another embodiment, the implantable composition comprises demineralized bone matrix in combination with one to five different siRNA molecules directed to more than one target. In another embodiment, an siRNA effector molecule targeting an protein inhibitor of chondrogenesis is incorporated into a pharmaceutically acceptable carrier or a bone paste implant material for implantation. Suitable pharmaceutically acceptable carriers are well known in the art. Suitable bone paste materials are commercially available under the tradenames OSTEOFIL®, OPTEFORM®, and REGENAFIL™ from Regeneration Technologies, Inc., Alachua Fla. In another embodiment, 1 to 5 different siRNA molecules targeting 1 to 5 different mRNAs that encode inhibitory polypeptides are incorporated into the pharmaceutically acceptable carrier or bone paste implant material.

In another embodiment of a composition of the present invention, 1 to 5 different siRNA effectors that are directed at different targets, respectively, are incorporated into a specially formulated bone paste implant material. Specially formulated bone paste products include, for example, carriers that enhance siRNA stability and/or cell-mediated uptake. These carriers include nanoparticulate carriers such as neutralized atelocollagen. Also within the scope of the instant invention are carriers such as, but not limited to, gelatin, collagen, glycerol, hyaluronic acid, chondroitin sulfate, polyethylene oxide, polyvinylypyrrolidone, polyvinyl alcohol, dextran or mixtures thereof. Pastes for use with the principles of the invention include, but are not limited to, allograft pastes (e.g., osteogenic pastes or chondrogenic pastes), carrier associated Growth Factors, carrier associated mineralized particles, morsellized skin or other tissue, Fibrin powder, Fibrin/plasminogen glue, biomedical plastics, Demineralized Bone Matrix (DBM)/glycerol, cortico cancellous chips (CCC), DBM/PLURONIC® F127, and DBM/CCC/PLURONIC® F127, human tissue/polyesters or polyhydroxy compounds, or polyvinyl compounds or polyamino compounds or polycarbonate compounds or any other suitable viscous carrier; or alpha-BSM®; or polyethylene oxide, polyvinvylpyrrolidone, polyvinyl alcohol, collagen and dextran. Preferably, pastes used in accordance with the principles of the subject invention are graft pastes having osteogenic or chondrogenic properties. Furthermore, the paste components can include other materials such as, but not limited to, antibiotics, sucrose, dextrose or other biologically compatible anti-caking agents, and optionally, barium, iodine, or other high atomic weight elements for purposes of radioopacity. In another embodiment, the paste for use as taught herein contains a carrier, an osteoconductive component, and an osteoinductive component. Carriers can include, but are not limited to, gelatin, collagen, glycerol, hyaluronic acid, chondroitin sulfate, polyethylene oxide, polyvinylypyrrolidone, polyvinyl alcohol, dextran and/or mixtures thereof. Osteoconductive materials suitable for use with the subject invention include, but are not limited to, hydroxapatite (HA), tricalcium phosphate (TCP), CCC, bioactive glass, bioactive ceramics, and/or mixtures thereof. Osteoinductive materials suitable for use with the subject invention include, but are not limited to, DBM, and growth factors such as bone morphogenic protein (BMP), TGF-beta, PDGF, and/or mixtures thereof.

In yet another embodiment, a single (1) or multiple (2 to 5) siRNA effectors are incorporated into hard or soft tissue implants. Implants comprise cortical bone, cancellous bone, soft tissue, synthetic material or combinations thereof. Also within the scope of the invention are materials such as sponges, sheets or strips made of bone, soft tissue or combinations thereof. For hard or soft tissue implants, the siRNA molecule is physically (e.g. adsorbed) or chemically attached to the implant. Implants may be conventional forms (e.g. fibular ring, tricortical iliac block), or machined grafts and may be assembled from smaller pieces. Additionally within the scope of this invention is the selective treatment of a portion or portions of implants with siRNA effectors (single or multiple (2-5)). Suitable hard and/or soft tissue implants are commercially available from Regeneration Technologies, Inc. Alachua Fla., or are disclosed in the following U.S. patents and patent applications which are incorporated herein by reference: U.S. Pat. No. 4,950,296; U.S. Pat. No. 5,814,084; U.S. Pat. No. 6,033,438; U.S. Pat. No. 6,045,554; U.S. Pat. No. 6,096,081; U.S. Pat. No. 6,090,998; U.S. Pat. No. 6,290,718; U.S. Pat. No. 6,409,765; U.S. Pat. No. 6,497,726; U.S. Pat. No. 6,652,592; U.S. Pat. No. 6,685,626; U.S. Pat. No. 6,695,882; U.S. Pat. No. 6,699,252; U.S. Pat. No. 6,805,713; U.S. Pat. No. 6,893,462; U.S. Pat. No. D461,248; Ser. Nos. 10/793,976; 10/754,310; 10/387,322; 09/722,205; 09/701,933.

A preferred siRNA molecule for use in the above compositions and/or implants is a double stranded RNA of 5 to 40 bp (typically 13 to 30 bp, more typically 19 to 23 bp) that down-regulates the expression of SMURF-1 or HDAC-3. When the siRNA is directed against SMURF-1, the siRNA (sense strand) has the nucleotide sequence selected from the group consisting of SEQ ID NO: 1, SEQ ID NO: 3, SEQ ID NO: 5 and SEQ ID NO: 7; preferably, selected from the group consisting of SEQ ID NO: 1, SEQ ID NO: 3 and SEQ ID NO: 5; more preferably, selected from the group consisting of SEQ ID NO: 1 and SEQ ID NO: 3; and most preferably, the siRNA sense strand has the nucleotide sequence of SEQ ID NO: 1.

When the siRNA is directed against HDAC-3, the siRNA (sense strand) has the nucleotide sequence selected from the group consisting of SEQ ID NO: 9, SEQ ID NO: 11, SEQ ID NO: 13 and SEQ ID NO: 15; preferably, selected from the group consisting of SEQ ID NO: 9, SEQ ID NO: 11 and SEQ ID NO: 13; more preferably, the siRNA sense strand has the nucleotide sequence of SEQ ID NO: 9.

In another aspect, the present invention is directed to a method of down-regulating the expression in a mammalian cell of a protein that inhibits osteogenesis, comprising administering to the cell a double stranded siRNA molecule of 5 to 40 bp (more typically 13 to 30 bp, most typically 19 to 23 bp) that down-regulates the expression of a protein that inhibits osteogenesis. In a further aspect, the present invention is directed to a method of down-regulating the expression in a mammalian cell of a protein that inhibits chondrogenesis, comprising administering to the cell a double stranded siRNA molecule of 5 to 40 bp (more typically 13 to 30 bp, most typically 19 to 23 bp) that down-regulates the expression of a protein that inhibits chondrogenesis. Preferably, the siRNA molecule down-regulates SMURF-1 or HDAC-3. Typically, the mammalian cell of this method is an osteoblast or a pre-osteoblast. Suitable siRNA molecules for down-regulating SMURF-1 and HDAC-3 have the sequence already described above. Preferred siRNA molecules are also as already described above.

In yet another aspect, the present invention is directed to a method of enhancing osteogenesis in a mammalian patient, particularly a human patient, in need of treatment comprising administering to the patient at a site in need of osteogenesis an effective amount of a double stranded siRNA of 5 to 40 bp (more typically 13 to 30 bp, most typically 19 to 23 bp) that down-regulates the expression of a protein that inhibits osteogenesis. In a further aspect, the present invention is directed to a method of enhancing chondrogenesis in a mammalian patient, particularly a human patient, in need of treatment comprising administering to the patient at a site in need of chondrogenesis an effective amount of a double stranded siRNA of 5 to 40 bp (more typically 13 to 30 bp, most typically 19 to 23 bp) that down-regulates the expression of a protein that inhibits chondrogenesis. Preferably, the siRNA molecule down-regulates SMURF-1 or HDAC-3. Suitable siRNA molecules for down-regulating SMURF-1 and HDAC-3 have the sequence already described above. Preferred siRNA molecules are also as already described above.

In an additional embodiment, siRNA molecules are used for the treatment of damaged intervertebral discs by replenishment of the nucleus pulposus (nucleus pulposa). To date, no procedure and/or device is available that can restore functionality and mobility to a damaged spine segment through the regeneration of disc tissue. Simply replacing the damaged tissue or increasing the number of cells is insufficient to inhibit degeneration. The extracellular matrix components of the disc actually provide the structural integrity important for sustaining the tensile and torsional forces imposed on the spine. The integrity of the disc is compromised when loss of water content in the nucleus or changes in tissue composition (proteoglycan versus fibrocartilage) occurs through damage or disease.

Disc damage and/or degeneration are accompanied by an inflammatory effect; the presence of the accompanying cytokines in the matrix inhibits repair. As long as an inflammatory state exists, macrophages will continue to produce proteins that are detrimental to the extracellular matrix of the nucleus pulposus. Thus the object of this invention is to allow for nucleus pulposus regeneration by removing any inhibitory elements that are associated with disc damage and thus allow for successful rebuilding of the extracellular matrix.

Molecules resulting from inflammation contribute to the breakdown of the extracellular matrix of the nucleus pulposus. These molecules include, but are not limited to, IL-1, IL-6, PGE₂, NO and TNF-α. Additional inhibitory elements include, but are not limited to, the following catabolic factors (degradative enzymes) MMP-1, MMP-3, iNOS and IL-1β. Treatments with siRNA (or anti-sense RNA) would prevent the translation of these proteins from their mRNA templates by binding to the mRNA template and prevent it from contributing to protein production. Sequences are determined or obtained from databases and used in the construction of either siRNA or anti-sense RNA.

Delivery of siRNA targeted to molecules responsible for inflammation and matrix breakdown shuts down their production, allowing for the creation of a favorable environment in the disc for cell viability, proteoglycan production, and extracellular matrix integrity. Specifically, macrophages are isolated from blood and induced to produce cytokines after which the siRNA constructs are introduced and the presence of inflammatory molecules monitored via PCR or ELISA. In vivo models of disc degeneration (5 mm puncture model) are used to determine the impact of siRNA constructs on reducing disc inflammation at the disc site (via immunohistochemistry).

### BRIEF DESCRIPTION OF SEVERAL VIEWS OF THE DRAWINGS

FIG. 1 is a view of agarose gels demonstrating siRNA-mediated gene-specific knockdown using SMURF-1 and HDAC-1 reagents. MC3T3 (mouse pre-osteoblasts) and D1 (mouse multipotent bone marrow stromal cells) cells were separately transfected with each of 4 different siRNAs (Qiagen) representing each target gene and mRNA was then prepared two days later. Equal 0.5 ug aliquots of each mRNA were amplified with either a SMURF-1 or HDAC-3 primer pair along with GAPDH primers as an equalization control (rows labeled GAPDH). Lanes C represent duplicate control (scramble-transfected) cell cultures and each of the 4 SMURF (S) and HDAC (H) cultures are signified by numbers 1-4.

FIG. 2 is a view of agarose gels of Q-PCR (qRT/PCR) products generated from mRNAs isolated from MC3T3 and D1 cells transfected with either a scrambled siRNA as a control (cont.) or HDAC-3 siRNAs (siRNA). Samplings were performed on day-2 (d2) and day-6 (d6) following transfection. The PCR primers used to generate these volume-equalized samples (0.5 ug mRNA/Q-PCR (qRT/PCR) reaction) are indicated on the left.

FIG. 3 is a view of agarose gels of Q-PCR (qRT/PCR) products generated from mRNAs isolated from MC3T3 and D1 cells transfected with either a scrambled siRNA as a control (cont.) or SMURF-1 siRNAs (siRNA). Samplings were performed on day-2 (d2) and day-6 (d6) following transfection. The PCR primers used to generate these volume-equalized samples (0.5 ug mRNA/Q-PCR (qRT/PCR) reaction) are indicated on the left.

The foregoing summary, as well as the following detailed description of certain embodiments of the present invention, will be better understood when read in conjunction with the appended drawings. For the purpose of illustrating the invention, there is shown in the drawings, certain embodiments. It should be understood, however, that the present invention is not limited to the arrangements and instrumentality shown in the attached drawings.

### DETAILED DESCRIPTION OF THE INVENTION

The instant invention relates to bone grafting, mediators of BMP signaling, and siRNA. The design of implants that take advantage of BMP signaling have had perhaps the greatest clinical and commercial impact of any orthopedic devices in the past five years. BMP-2 and BMP-7 have been shown to be excellent at inducing bone regeneration and several products have proven their efficacy in animal (Liao et al., 2003) and human (Boden et al., 2002) clinical trials where posterolateral lumbar fusion (PLLF) has been achieved using recombinant BMP-2. BMP-2 is delivered in a collagen sponge (InFuse®) inserted into an implantable titanium cage, resulting in fusion rates approaching 100% in a pivotal study of 250 recipients. A PMA for this device was awarded in July 2002. BMP-7 (OP-1) has been reported to be 50-70% successful in inducing PLLF in human studies conducted to date. This OP-1 product (Stryker Biotech) has subsequently received Humanitarian Device Exemption (HDE) status in late 2001 from the FDA for use in treating recalcitrant long bone non-union fractures. OP-1 putty has also received HDE status. OP-1 Putty is indicated in Revision Spinal Fusions where autograft is unfeasible.

Despite these favorable outcomes, the underlying technologies are nonetheless reliant on delivering massive amounts of BMP cytokine to anatomic regions requiring bone regeneration in order to up-regulate BMP signaling pathways. This approach adds cost (thousands/implant for InFuse®) to already expensive procedures. Also, clinical experience over the next few years will be required to unequivocally demonstrate safety as well as efficacy. Indeed, third party reimbursement policies regarding these devices deem cytokine usage contraindicated in persons with known hypersensitivity, in skeletally-immature individuals, in pregnant women, and in persons with a history of malignancy. In addition OP-1 is considered by the FDA to be “experimental and investigational” for use in spinal fusion and that it is not medically indicated if intended to be applied at the site of resected tumors (Aetna Clinical Policy Bulletin #0411).

Demineralized bone matrix (DBM) has been available for clinical use for over 10 years. DBM is a manufactured product consisting of demineralized, pulverized cortical bone that largely retains the original osteoinductive cytokines that were deposited in the bone matrix during the donor's lifetime. Studies (Edwards et al., 1998; Wang and Glimcher, 1999; Takikawa et el., 2003) have confirmed the osteoinductivity of DBM using implants grafted at both ectopic (muscle pouch) and calvarial (cranium) sites. Significant new bone (calvarial sites) or both cartilage and bone (ectopic site) are induced by DBM, consistent with laboratory studies demonstrating release of cytokines by collagenases that mimic natural bone remodeling processes mediated by matrix metaloproteases. Clinically-relevant levels of osteoinductive cytokines including BMP-2, BMP-4, TGF-beta 1 and TGF-beta 2 are present in commercially available DBM. It is also important to note that DBM paste products also provide an osteoconductive surface (i.e., promoting the attachment of osteogenic cells) for cell binding that foreign substrates such as bovine collagen sponges poorly mimic. However, in spite of the advantageous properties of DBM, the need for improved products exists. The Applicants discovered_that the osteogenic signal generated by BMPs in DBM, when amplified using inhibitor-degrading siRNAs, results in a composite graft with increased bone-forming properties.

Early studies that relied upon in vivo delivery of siRNA were hampered by both the inherent sensitivity of RNA to serum nucleases and to poor uptake by target cells. Several novel stratagems have been developed over the past few years to circumvent these problems. These run the gamut from physical methods that utilize high pressures and large injection volumes for systemic delivery (Lewis et al., 2002), covalent modifications of siRNA molecules (most notably attachment of cholesterol to the 3′ end of the sense strand; Soutschek et al., 2004), siRNAs formulated with protamine-conjugated antibodies (Song et al., 2005), and encapsulation of siRNAs within atelocollagen nanoparticles (Minakuchi et al., 2004).

As a result of these advances siRNA technologies have emerged as a clinically-viable route to treat diseases. A new paradigm for the treatment of cardiovascular disease through the suppression apoB protein expression has recently been demonstrated via the systemic injection of cholesterol-derivatized siRNA (Soutschek et al., 2004). Underivatized siRNA (directed against VEGF mRNA) when delivered intraocularly has been shown to be effective in reducing the severity of macular degeneration (Reich et al., 2003) and at least two such drugs are in Phase I clinical trials. Treatment of diseases in which siRNA can be delivered intranasally such as viral influenza and respiratory syncytial virus show promise as well (see Shankar et al., 2005).

The Applicants discovered that rates and extents of bone healing can be increased through targeting negative regulators. In vitro studies (in which several genes regulating dissimilar aspects of BMP signaling are targeted) reveal both cell-type specific siRNA molecules for clinical use and combinations of siRNA molecules that synergize with each other to enhance knockdown. Delivery with a DBM paste vehicle in combination with application in specific protected sites affords higher efficiencies of knockdown than has been achieved with applications requiring systemic injection. The instant invention also has applications for enhancing new cartilage formation via siRNA molecules owing to both clinical need for cartilage regeneration as well as recognition of endochondral bone formation which takes place upon a mineralized cartilage model (Ferguson et al., 1999; Gerstenfeld et al., 2003).

Target Genes Encoding Inhibitory Proteins

Increases in knowledge of the genes regulating bone formation have taken place over the past 5 years. In particular, the genes regulating BMP signaling pathways (both positively and negatively) have been cloned, characterized structurally and functionally, and their integrative roles in bone formation relative to other gene products determined. These inhibitory genes were chosen by Applicants to represent proteins that interact with significant members of BMP signaling including BMPs themselves, SMADS and runx2. Major categories of negative regulators of BMP signaling include, but are not limited to, the following:

Runx2 binding proteins: The STAT1 gene has been shown to bind to and inhibit runx2, the primary transcriptional activator of osteoblast differentiation. STAT1 knock-out mice exhibit increased bone and osteoid mass, and explanted pre-osteoblasts from null lines display enhanced responsiveness to BMP-2 as evidenced by increased alkaline phosphatase activity, elevated levels of osteopontin and osteocalcin mRNA, and accelerated rates of nodule mineralization. Runx2 promoter-binding activity is also enhanced in mutants. Significantly STAT1 functions post-natally to inhibit bone formation (Xiao et al., 2002; Kim et al., 2003).

Extracellular BMP binding proteins: Noggin is an excreted 64 kD glycoprotein that binds to extracellular BMP-2, inhibiting its binding to receptor (Abe et al, 2000; Yoshimura et al., 2001). Noggin is expressed in pre-osteoblasts that have not been reexposed to BMP and thus seems to serve a role in maintaining developmental-stage stasis (Gazzero et al., 1998). Noggin remains in the matrix of processed DBM and its selective removal enhances osteoinductivity (Behnam et al., 2004). Genes encoding proteins functionally-identical to Noggin include Cerberus (Piccolo et al, 1999), chordin (Piccolo et al, 1996), DAN (Stanley et al, 1998), gremlin (Hsu et al, 1998), sclerostin (vanBezooijen et al, 2002), twisted (Ross et al, 2001), and ventropin (Sakuta et al, 2001).

SMAD Ubiquitinating Proteins: SMURFs (SMAD-ubiquitinating regulatory factors) comprise a small family of key proteins that ubiquitinate receptor-activated SMADs thereby targeting them for degradation (Ying et al., 2003). C2Cl2 muscle cells express high levels of SMURF proteins and their overexpression in these cells prevents the BMP-mediated transdifferentiation into osteoblasts. Similarly, osteoblasts transfected with SMURF1 constructs exhibit reduced alkaline phosphatase activity and lower levels of runx2, osteocalcin, and osterix mRNAs (Zhao et al., 2004). This family includes, but is not limited to, SMURFs 1, 2, and 3. A related protein is CHIP, which mediates SMAD ubiquitization via another mechanism (Li et al, 2004).

Transcriptional Attenuators: HDACs (histone deacetylases) are nuclear-localized regulatory enzymes which inactivate the expression of specific genes through histone deacetylation. HDAC-3 binds specifically to the runx2 transcriptional complex leading to a loss of promoter binding and transcriptional activation. Inhibition of HDAC-3 expression in MC3T3 osteoblasts using siRNA culminates in accelerated nodule formation/mineralization and expression of osbeoblast-characteristic genes such as osteocalcin (Schroeder et al., 2004).

SMAD Inhibitors: Tob is a member of an anti-proliferative gene family. Tob knock-out mice have no phenotypic abnormalities other than enhanced bone mass which by 9 months exceeds that of normal mice by 250%. This phenotype is due to increased numbers of osteoblasts in Tob−/− mice and appears to be the result of the relief of Tob inhibition of BMP signaling which is manifested by binding of Tob with SMADS which relegates them to localization in inactive “nuclear bodies” (Yoshida et al., 2000). Other proteins in this category include BMP-3 (an antagonist of SMAD activation; Daluiski et al, 2001) and SMADS-6 and -7 (interfere with BMP-mediated signaling via the TGF-beta signaling pathway; Miyazono, 1999).

BMP Signaling inhibitors: Calponin is a smooth muscle cell differentiation factor that carries out its function through binding to alpha-smooth muscle cell actin and SMADS. Knock-out mice that are calponin−/− exhibit higher than normal ectopic bone formation in muscle tissue when exposed to BMPs. Similarly, osteoblasts isolated from calponin−/− animals have elevated bone markers such as alkaline phospatase (Yoshikawa et al., 1998). Other genes encoding inhibitory products include BAMBI (a BMP psuedoreceptor that blocks binding; Onicktchou et al, 1999) and Notch receptors 1-4 (interfere with the kinase activity of BMP receptors; Nobta et al, 2005), and msx2 (accelerates BMP-mediated osteoblast apoptosis; Marazzi et al, 1997).

Mineralization Inhibitors: S100A4 is a protein found in periodontal ligament that prevents the mineralization of this tissue. The same protein is also expressed in osteoblasts and its inhibition using siRNA has been shown to markedly enhance osteoblastic characteristics (Kato et al, 2005).

The genes encoding proteins that inhibit osteoblastic activity include, but are not limited to the targets listed above. Preferably, the targets genes encode a protein selected from the group consisting of HDAC-3 (a negative co-repressor that binds to runx2); STAT-1 (which likewise is a runx2-binding protein); SMURFs-1, -3 and -8 (SMAD-ubiquitinating proteins); Tob and calponin (both of which are SMAD binding proteins that interfere with SMAD nuclear localization); and noggin (a bone matrix-localized, extracellular protein inhibiting the interaction of BMP with its receptor) for a total of eight genes. Applicants and others (Schroeder et al., 2004; Kato et al., 2005) have demonstrated that a measurable enhancement of osteoblastic characters accompanies transfection of a single potent siRNA complementary to inhibitor mRNAs. Thus one embodiment of the instant invention is directed to a siRNA molecule capable of down-regulating osteo-inhibitory gene products and/or enhancing bone-forming gene expression by reduction of levels of inhibitory proteins. Another embodiment is directed to a combination of siRNA molecules targeting two or more of the candidate genes simultaneously. In yet another embodiment, the instant invention is directed to implant materials that comprise a combination of different siRNA molecules.

siRNA transfections of cell cultures exposed to sub-threshold (i.e., 50 ng/ml or less) amounts of recombinant BMP-2 (Sigma) required to elicit osteoblast markers demonstrate that the relief of specific inhibitory steps amplifies de novo BMP signaling. siRNAs for the selected genes are designed and then synthesized using algorithms developed by suppliers (e.g. Qiagen (Valencia, Calif.)). Double-stranded 21-mers (with 3′ dTdT and dTdG overhangs) are used, although it is also within the scope of the instant invention to use blunt-ended 27-mers synthesized (based on successful 21-mer sequences) to evaluate increased knockdown as others have had success using these constructs (Kim et al., 2005). The mouse cell lines used for transfections include, but are not limited to, C2Cl2 myoblasts cells, D1 multipotent bone marrow stromal cells and MC3T3 pre-osteoblasts. These cell lines provide a good representation of cells likely to be affected by siRNA transfection in vivo including fully-differentiated cells (muscle, represented by C2Cl2 cells), uncommitted mesenchymal stem cells (D1 cells) and committed but undifferentiated osteoblasts (MC3T3 cells). Human cell lines used for transfections include, but are not limited to, SAOS-2 osteosarcomea cells, MG63 preosteoblast, ATDC5 prechondrocytes, and HGF gingival fibroblasts. The rat cell lines used for transfections include, but are not limited to, rat calvarial osteoblasts. It is also within the scope of the instant invention to carry out transfections using primary cell lines derived from calvaria (osteoblasts), stem cells (e.g. adipose derived), and bone marrow aspirates (multipotent mesenchymal stem cells) to verify both delivery of siRNAs and knockdown.

PCR primers complementary to the mRNAs targeted for knockdown are synthesized (GenoMechanix) and RT-PCR (SybrGreen RT-PCR Kit, Qiagen) is used to verify expression in test cell lines. Determination of the most effective amount of siRNA and volume of transfection reagent is conducted using the appropriate cell lines. Concentrations of siRNA in the range of about 0.1 nM-50 nM are used. Cells for transfection are set up in triplicate for analyses. Messenger RNA isolated from cells transfected with siRNAs (0.25-0.5 ug, 24 well format) is subjected to real time Q-PCR (qRT/PCR) (iCycler, BioRad) to establish the degree of knockdown as compared with mock-transfected cells (transfected with a “scrambled” siRNA). Threshold detection values (“Ct values”) are generated using PCR primers directed against a housekeeping protein (GAPDH) and comparable data from the target mRNAs is compared to calculate the % knockdown. Data is verified by comparing aliquots of reaction products run on agarose gels stained with ethidium bromide. RNA from cells processed 2, 4, 6, and 8 days following siRNA transfection are analyzed to establish the duration and extent of knockdown. Acceptable reductions are greater than about 50%, preferably greater than about 60%, more preferably about 75-99%.

Osteoinduction induced by target mRNA knockdown is the emergence of BMP signaling-dependent osteoblastic phenotypes not present in the parent, untransfected cells. Cell extracts are prepared to quantify alkaline phosphatase specific activity, an early marker of osteoblast differentiation. A spectrophotometric assay using p-nitrophenol phosphate as substrate is used. Levels of osteoblastic “bone marker” mRNAs are measured by Q-PCR (qRT/PCR). These include, but are not limited to, runx2, bone sialoprotein, osteocalcin, osteopontin, typeI collagen, and osterix. Previous studies of this type using either BMPs, bone-inducing growth factors (such as dexamethasone, vitamin D12) or recombinant adenovirus-expressed BMP or runx2 to increase osteoblastic characters in cultured cells, have demonstrated that 2-5-fold increases in both alkaline phosphatase and the bone markers are the minimal thresholds for successful osteoblastic differentiation (Prince et al., 2001; Viereck et al., 2002; Yang et al., 2003; Jorgensen et al., 2004). Also, some markers such as osteocalcin are not be expressed in either the undifferentiated or non-osteoblastic cells but are induced de novo if siRNA knockdown enhances BMP signaling and is thus osteoinductive. Quantitative data (measured Ct values) is corroborated by agarose gel electrophoresis of the PCR reaction products.

runx2 serves as a marker of osteoblastic differentiation with a defined role as a key transcriptional co-activator in bone-forming cells (Karsenty et al., 1999; Komori, 2000). Thus Applicants used a functional assay for the runx2 protein utilizing a luciferase gene (pGL-3, Promega) under the control of six, tandemly-repeated osteocalcin gene enhancer elements (“6×OSE”; Ducy and Karsenty, 1995; Ducy et al., 1997). For these studies the reporter plasmid is either co- or post-transfected with siRNAs. Extracts are prepared from transfected cells after a set period of time (e.g. 1-2 days) and luciferase activities are determined using a luminometer assay (Promega). As is the case with other quantifiable osteoblast phenotypes, relief of BMP signaling inhibition enhances runx2 enhancer activation (as detected by enhanced luciferase activity) 2- to 5-fold.

It is also within the scope of the instant invention to evaluate BMP signaling enhancement through siRNA knockdown in one or both of the following two manners. The first is an assessment of the ability of siRNA-transfected cells to undergo matrix mineralization in the presence of osteoblast-enhancing supplements (ascorbic acid, beta-glycerophosphate and dexamethasone). Undifferentiated C3H10T1/2 mesenchymal cells, for example, undergo mineralization only when exposed to BMP or when transduced with BMP-expressing adenovirus stocks (Yang et al., 2003). Accordingly, siRNA-transfected cell lines showing highly induced levels of osteoblast transcripts are grown to confluence, exposed to osteoblastic stimulants, and then vonKossa staining used to detect mineral deposition two-three weeks later. The second is assessment of reprogramming of differentiated cell lines using PCR primers complementary to gene products.

Thus, for example, a decline in myogenin and myoD is seen for in siRNA-transfected C2Cl2 muscle cells (Katagiri et al., 1994; Ying et al., 2003; Zhao et al., 2004) and lipolipoprotein and adipcin transcripts are less abundant in siRNA-treated D1 mesenchymal precursor cells (Zuk et al., 2001; Li et al., 2003). Applicants have shown that C2Cl2 and D1 cells abundantly express myogenin/myoD and adipcin/lipolipoprotein, respectively.

The instant invention is also directed to a siRNA delivery system (implant material). An effective delivery system must satisfy the following criteria: provides reasonable resistance to nucleolytic degradation; is well-retained at the site of implantation; facilitates the uptake of siRNA by cells populating the implant; and possesses favorable pharmacokinetic/release properties. In one embodiment of the instant invention, a single siRNA effector molecule targeting an osteoblast-inhibiting gene is incorporated into a commercially available bone paste implant material for implantation at bony defects. In another embodiment, siRNAs targeting multiple mRNAs encoding inhibitory polypeptides are incorporated into the implant material. In another embodiment, a single or multiple siRNA effectors are incorporated into a specially formulated bone paste implant material. Specially formulated bone paste products include carriers that enhance siRNA stability and/or cell-mediated uptake. These carriers include nanoparticulate carriers such as neutralized atelocollagen. In yet another embodiment, a single or multiple siRNA effectors are incorporated into machined cortical or cortical-cancellous bone implants. For machined cortical or cortical-cancellous bone implants, siRNA molecules are physically (e.g. adsorbed) or chemically attached to the implant.

Also within the scope of the present invention are siRNA molecules that have been themselves chemically or physically modified. A non-limiting example of chemical modification is cholesterol-derivatization and a non-limiting example of physical modification is formulation with atelocollagen. siRNA covalently linked to cholesterol has been reported to exhibit levels of knockdown comparable to underivatized molecules and at lower concentrations (3 nM versus 200 nM). The in vivo half lives for cholesteroyl-siRNAs were correspondingly increased from 6-95 minutes, presumably due to complexation with serum albumins (Soutschek et al., 2004). Acceptable reductions are knockdown of 75-95% with a minimal 2 to 5-fold up-regulation of osteoblastic phenotypes.

Atelocollagen is fibrillar collagen digested with pepsin to remove N- and C-terminal peptide fragments, producing a product that is soluble at low pH and 4 C but that forms a gel at neutral pH and 37 C. In one embodiment, human tendon from donor tissue (comprised principally of typeI collagen) is digested for 3 days in 15 mM HCl containing 0.05 mg/ml pepsin (type I, Sigma) at 4 C. Following a low speed centrifugation to remove undigested tissue, the pH of the clarified supernatant is adjusted to 10 to inactivate pepsin and then down to 7.2 to precipitate atelocollagen. The atelocollagen is taken through 2 additional cycles of solubilization/precipitation (Ochiya et al., 1999; Lee et al., 2004). To form a nanoparticulate preparation, equal volumes of neutralized atelocollagen (0.008% and siRNAs (380 ng/ml) is mixed and 50 ul aliquots spotted into the wells of 24 well plates (Minakuchi et al., 2004). Appropriate cells are plated on top of the atelocollagen-siRNA complex and knockdown/osteoblastic enhancement studies conducted and evaluated. For animal studies atelocollagen and siRNAs are mixed, lyophilized, rehydrated, and extruded as a gel for implantation as described (Minakuchi et al., 2004).

Animal studies validate stability and effectiveness of siRNAs using a luciferase reporter system to monitor the extent and duration of knockdown in siRNA-containing implant materials. A luciferase reporter plasmid containing a hygromycin resistance gene (pGL4/hygro, Promega) is transfected into either an established cell line or into cells from bone marrow aspirates prepared from the femurs of sacrificed rats. Cell lines stably-transfected with this plasmid are recovered over the course of 10 cell doublings using 0.5-1 mg/ml hygromycin. A siRNA directed against the luciferase reporter is synthesized and knockdown validated in in vitro transfections. Stably-transfected cells are added to DBM implants (with or without siRNA), implanted into an ectopic site in athymic nude rats, and luciferase activity monitored in explants over time using a luminometer-based assay (Luciferase Assay System, Promega). Measurable luciferase enzyme activity is induced in explants for at least a few days following implantation. Indeed, proteins encoded by adenovirus-transduced osteoblasts (Yang et al., 2003) and fibroblasts (Hirata et al, 2003) continue to be expressed at either ectopic or calvarial sites of implantation for at least a week. Similarly, siRNA delivered either systemically by high-pressure injection or by localized injection have been shown effective in suppressing plasmid- or tumor-encoded luciferase activity in rats (Lewis et al., 2002; Takei et al., 2004). Diminution of luciferase activity in our model demonstrates effective siRNA delivery (either by cholesterol conjugation or atelocollagen encapsulation), enhanced siRNA stability, effective release, and then device-mediated uptake of siRNA by stably-transfected cells.

The magnitude of knockdown, its duration, correlation with delivery vehicle, and specificity (as compared with “scrambled” siRNA controls, mock transfected cell implants, and the like) is measured. siRNAs are delivered in the range of about 1-500 ug/implant; preferably about 5-200 ug/implant; more preferably about 10-100 ug/implant for animal studies. Initial animal studies are conducted using an ectopic model (athymic nude mice) for bone/cartilage formation monitor knockdown efficacy and allow for identification of enhanced rates, duration and/or magnitude of cellular events characteristic of new bone formation. Further animal studies use a closed fracture repair model (long bone) and healing of experimental calvarial defects (Einhom, 1999; Wang and Glimcher, 1999; Chesmel et al., 1998). For these studies and for human delivery, siRNAs are delivered in the ranges listed above or in the range of about 1-1000 ug/implant; preferably about 20-800 ug/implant; more preferably about 50-500 ug/implant. In vivo siRNA delivery systems incorporating antibody targeting (Song, 2005) in place of or in conjunction with other procedures such as atelocollagen nanoparticle encapsulation and/or cholesterol-derivatization are also within the scope of the present invention.

Demineralized bone matrix (DBM)-based paste products currently used as general orthopedic grafts show useful bone repair properties, although are limited in their clinical scope. An embodiment of the instant invention is directed to improved DBM-based implant materials. In one embodiment of the instant invention, a single siRNA effector molecule targeting an osteoblast-inhibiting gene is incorporated into a commercially available bone paste implant material for implantation at bony defects. In another embodiment, siRNAs targeting multiple mRNAs encoding inhibitory polypeptides are incorporated into the implant material. In another embodiment, a single or multiple siRNA effectors are incorporated into a specially formulated bone paste implant material. Specially formulated bone paste products include carriers that enhance siRNA stability and/or cell-mediated uptake. An ectopic model using athymic nude rats or mice (rats preferred) in conjunction DBM incorporating with siRNA allows for the measurement of new cartilage, bone and/or marrow formation. The ectopic model used to assess the osteoinductivity of DBM preparations is well-characterized and provides a framework within which the events accompanying endochondral bone formation are recapitulated and easily-analyzed (Bessho et al., 1992; Edwards et al., 1998). These events are triggered by cytokines released from DBM during metalloprotease-induced remodeling of the graft such as TGF-betas (osteoblast replication), BMPs (osteoblast differentiation), vascular endothelial growth factor (vascularization), cartilage-derived morphogenetic proteins (cartilage formation) among others.

Bone paste products are biomimetic scaffolds that have already shown clinical success. These materials serve as a scaffold for the delivery of siRNAs to assist in the bone regenerative process. The DBM implants used in our animal studies comprise clinical-grade, demineralized, freeze-dried bone powder containing cortical bone in the size range of 125-180 um. This material, when combined with siRNA in sterile, RNAase-free water, promotes new bone formation, remains localized at the implant site, provides a matrix for retaining the delivered reagent (especially in the case of relatively insoluble atelocollagen precipitates), and will provide an osteoconductive surface hospitable to the colonization of cells that are candidates for transfection. DBM that has been extracted with 4M guanidine-HCl and then rinsed is used. This treatment effectively removes all osteoinductive cytokines from bone matrix rendering the DBM non-osteoinductive (Sampath et al., 1987). Thus, the positive effects of siRNA for enhancing new bone (osteogenesis) or cartilage (chondrogenesis) formation are readily apparent and measurable on an osteoinductive baseline of “0”. DBM material previously determined to have low osteoinductivity scores (i.e., 1 or 2) is also used in comparative tests to demonstrate enhancement of the OI score due to siRNA delivery.

Implants prepared under sterile conditions comprise 10-100 mg DBM (preferably 20-55 mg) resuspended in 50-500 ul sterile water (preferably 50-100 ml) containing between 1 and 100 ug siRNA (cholesterol-derivatized or atelocollagen formulated). In vivo siRNA studies published to date indicate that as little as 50-80 ng are required either systemically or at confined tumor injection sites (Lewis et al., 2002; Takei et al., 2004). A more precise and experimentally-relevant amount of siRNA delivered is determined from luciferase knockdown studies. For each siRNA trial, identical paired experimental grafts is implanted contralaterally at an ectopic site. Duplicate rats are prepared for a total of 4 siRNA-containing rats per treatment. The remaining 2 implant sites in each rat receive the appropriate DBM without siRNA or DBM containing a scrambled version of the siRNA sequence used. Control grafts containing fully inductive DBM, cholesterol, and atelocollagen are also within the scope of the instant invention.

Anesthetized athymic nude rats are prepared for implantation by making a mid-ventral incision below the sternum followed by blunt dissection of the recti abdomini. Each of six prepared grafts (approximately 100 ul each) are introduced into the pocket created by the incision and upon completion the pocket will be closed with wound clips. Applicants have determined that the sequence of remodeling of the graft follows the general form of hematoma formation (day 1), cartilage formation (day 7), vascularization (day 10), bony ossicle formation (day 14) and/or marrow development (day 14). The DBM graft is completely enveloped in a capsule of fibrous tissue within 2 days of implantation making recovery and removal of an intact explant possible. Thus rats are sacrificed for explant analyses on days 14 and 28 post-implantation for assessment of the extent of significant remodeling milestones using histological techniques. It is also within the scope of the instant invention to sacrifice animals on shorter time basis (i.e., days 2, 4, 6, and 8) for biochemical analyses of the cells recruited to the implant site.

For histochemical studies the explants are placed in 10% buffered formalin for fixation and sections prepared for staining hematoxalin/eosin and Masson's trichrome reagents. At least three sections per implant (6 implants/rat) are evaluated by a blinded reader who assigns a score of from 0-4 for implants based upon the number of features seen in the sections that are consistent with osteogenesis. A second number (from 0-4) is assigned based upon visible evidence for inflammation. DBM samples inactivated with chaotropic reagents produce OI scores of 0,1 or 0,2. When osteogenesis is induced at the ectopic site for implant materials containing siRNA molecules, enhanced OI scores such as 1,1-3,1 are seen. Implants containing low OI rather than “0” OI DBM samples have commensurately increased scores as a result of siRNA delivery. Applicants believe that the signal provided by small amounts of BMPs (and perhaps other contributing cytokines) is amplified through the reduction of inhibitory molecules.

In another embodiment, biochemical analyses are used to test the extent to which siRNA mediates new bone formation at the ectopic site (in addition to or in place of histochemical evaluation). Biochemical criteria are more sensitive (as compared with the relatively demanding and remodeling sequence-dependent ossicle formation) for evaluation of siRNA-containing explants. These studies comprise, but are not limited to the following assays: alkaline phosphatase specific activity (spectrophotometric assay), bone marker mRNA titers (Q-PCR (qRT/PCR)), and 6×OSE-luciferase plasmid transfection of cells harvested with the bulk explant (luminescence assay).

Also within the scope of the present invention is the incorporation of a single or multiple siRNA effector molecules into other implant materials. Implant materials include, but are not limited to, machined cortical or cortical-cancellous bone implants of various shapes and sizes (for example, cortical or cancellous bone blocks, chips, dowels or pins). For machined cortical or cortical-cancellous bone implants, siRNA molecules are physically (e.g. adsorbed) or chemically attached to the implant. Additionally within the scope of the present invention is incorporation of siRNA reagents into implant materials by taking advantage of pressure-facilitated infiltration of bone. It is preferable to utilize the assignees' well known method of tissue treatment by alternating cycles of pressure and vacuum. These processes are disclosed in full detail in assignee's U.S. Pat. No. 6,613,278, entitled “Tissue Pooling Process,” which issued to Mills et al., on Sep. 2, 2003; U.S. Pat. No. 6,482,584, entitled “Cyclic implant perfusion cleaning and passivation process,” which issued to Mills, et al. on Nov. 19, 2002; and U.S. Pat. No. 6,652,818, entitled “Implant Sterilization Apparatus,” which issued to Mills et al., on Nov. 25, 2003, all of which are incorporated herein by reference in their entirety.

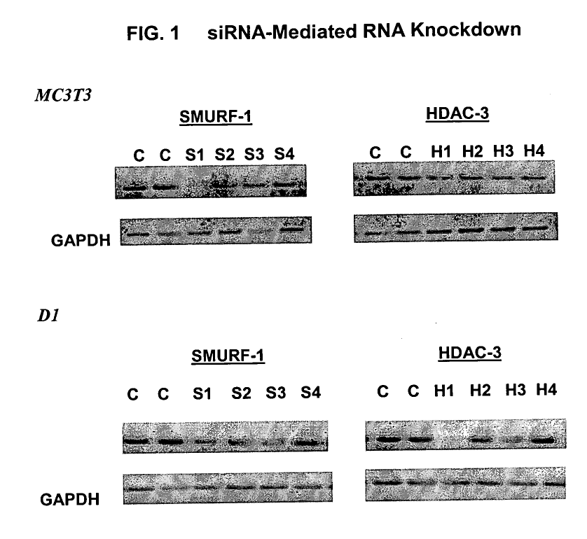

Studies for inducing knockdown of candidate inhibitory genes as well as enhancing osteoblastic phenotypes utilized siRNAs encoded as “hairpin” molecules used commercially-available plasmids (Promega). These plasmids were introduced into cells to establish stably-transfected sub-lines using geniticin as a dominant selectable marker. These studies were repeated on a larger scale by directly transfecting double-stranded siRNA since plasmids are inefficiently taken up by cells in vivo. siRNA knockdown sets comprised of 4 algorithm-designed sequences directed against SMURF-1 and HDAC-3 were synthesized by a commercial supplier (Qiagen). Members of both sets were introduced into MC3T3 pre-osteoblasts, C2Cl2 myoblasts, and D1 mesenchymal precursor cells using HiPerFect Reagent (Qiagen) and the extent of target gene mRNA ablation assessed using RT-PCR analysis. Agarose gels of the reaction products (FIG. 1) confirmed that the SMURF-1 siRNAs reduced their cognate mRNA by 2-fold (S4) to 10-fold (S1) in the two cell lines. Similarly, the HDAC-3 siRNAs reduced the corresponding message by 2- (H4) to 10-fold (H1) in the same cells. These results were typical of more extensive studies showing that the magnitude of knockdown achieved using a given siRNA is cell type-dependent (FIG. 1).

It was determined to be important to conduct knockdown tests in multiple cell types as well as evaluate the response of cells likely to be encountered at implant sites. It was found that the extent of residual knockdown was lower in 5-day cultures as compared with day-2 which can be attributed to continued cell replication which dilutes intracellular siRNA. While this effect confounds the interpretation of in vitro transfections, it was established that successful bone repair is not dependent on persistent mRNA knockdown. Rather, siRNA-mediated conversion of even a modest number of transfected cells to stably committed osteoblasts has a measurable outcome. Furthermore, the instant inventive concept of incorporation of siRNA into implant materials confers stable, long-term release of siRNA (for use in sites in which bone grafts are implanted). Additionally, applicants surprisingly discovered that every established cell line examined (whether or not committed or osteoblastic development) expresses at least SMURF-1 and HDAC-3 (FIG. 1), which shows that the potential for osteoblastic determination is latent in many more cell types than is generally appreciated.

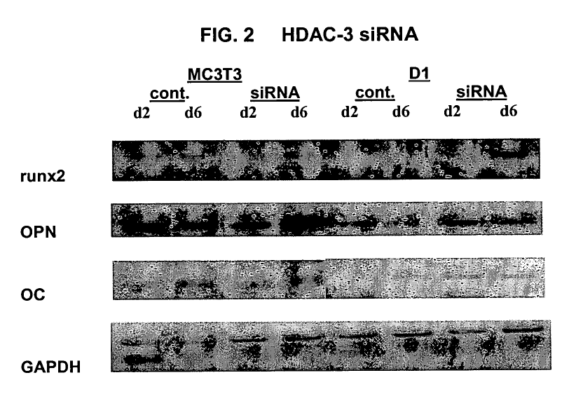

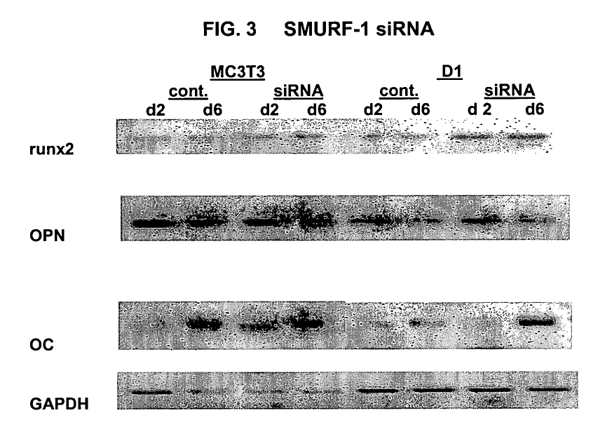

Representatives of the SMURF-1 and HDAC-3 siRNA collections yielding the highest degree of knockdown were used to evaluate concomitant changes in markers of osteoblast differentiation (FIG. 2 and FIG. 3). The key marker is expression of the runx2 promoter organizer, a gene that directly mediates BMP signaling at the level of de novo re-patterning of transcription (Ducy et al., 1999). In MC3T3 pre-osteoblasts HDAC-3 inhibition had little effect on runx2 transcript accumulation at either day-2 or day-6 post-transfection, while SMURF-1 siRNA enhanced mRNA accumulation 2-3-fold at both time points. By contrast D1 cells responded to both HDAC-3 and SMURF-1 siRNA transfection by increasing runx2 message by 32- and 14-fold at day-5 (respectively). The products of two other genes that are runx2-responsive, osteopontin (OPN) and osteocalcin (OC) showed comparable increases in transcript accumulation: MC3T3 cells showed 10-fold (HDAC siRNA) or 3-fold (SMURF siRNA) increases in OPN and 10-fold and 8-fold increases in OC; D1 cells responded with substantially similar patterns (FIG. 2 and FIG. 3).

In addition to the demonstration of increased transcript accumulation, other criteria must be met in order to verify that siRNA delivery enhanced BMP signaling at one or more levels. One of these is a direct assessment of runx2 function at the protein level since BMPs not only enhance transcription by promoting runx2 protein phosphorylation and translocation to the nucleus (Fujita et al., 2001; Katagiri and Takahashi, 2002; Stein et al., 2004). A straightforward test of runx2 function utilizes a reporter plasmid containing the following elements: a luciferase ORF; an SV-40 promoter; and a 6-fold tandemly-repeated promoter element from the osteocalcin gene (OSE-2) cloned upstream of the SV40 promoter (“6×OSE2” plasmid; Ducy and Karsenty, 1995). While luciferase activity is expressed in virtually any transfected cell type from the SV40 promoter, reporter activity is relatively greater in cells with a history of BMP signaling owing to increased runx2. In one such experiment it was found that MC3T3 cells that were stably-transfected with either SMURF-1 or HDAC-siRNA-expressing plasmids yielded 1.5 to 5-fold higher levels of luciferase as compared with untransfected controls (Table I). Even fully differentiated C2Cl2 myoblasts showed a greater than 2-fold increase in reporter activity when stably-transfected with HDAC-3 siRNA plasmid (Table 1).

Beyond these criteria for successful siRNA-mediated de- or re-differentiation, two additional criteria can be applied. Since only fully committed, terminally differentiated osteoblasts lay down hydroxyapatite in newly-excreted bone matrix, “mineralization” is an especially rigorous criterion (Hirata et al., 2003). One such experiment on relative degrees of mineralization in untransfected and siRNA transfected cells (using vonKossa staining for

Ca++deposition) showed accelerated mineralization of nodules in the latter as compared with the former. A second criterion that can be applied is the reduced accumulation of transcripts characteristic of a differentiated state other than “osteoblastic”. It has been found that myogenic-specific mRNAs such as myoD and myogenin were reduced several-fold in C2Cl2 myoblasts transfected with SMURF (1?/??) siRNAs. Similarly, robust lipolipoprotein and adipsin mRNA accumulation in D1 cells (poised to become adipocytes under appropriate culture conditions) was abolished by HDAC-3 siRNA transfection.

TABLE 1
Luciferase specific activities recovered from MC3T3 and C2C12 cells
transfected with a 6XOSE2 reporter plasmid. Indicated cells were
either untransfected (cont.) or stably-transfected with hairpin
plasmid vector encoding either a SMURF-1 or HDAC-3 siRNA.
Values shown are the mean activities from three
separate cell culture extracts (± SEM).
Luciferase Activity
(RLU/ug Protein)

| | |
|---|---|
| MC3T3 Cells | C2C12 Cells |

| Cont. | SMURF | HDAC | Cont. | HDAC |
|---|---|---|---|---|
| 209 | 307 | 1178 | 1258 | 2740 |
| (+30) | (+41) | (+86) | (+564) | (+117) |

### EXAMPLE 1

### Animal Studies

Athymic nude rats are used in these studies. The rat provides the lowest phylogenetic animal and the ectopic model described is relevant and acceptable for comparison to outcomes in humans. The rat model provides key information on new bone formation with siRNA prior to utilizing higher order animals. In terms of bone physiology and fracture repair a strong correlation has been established between the human and the rat. DBM grafts with or without siRNA are implanted in a muscle pouch created using a simple surgical procedure which will then be removed for further analyses at appropriate intervals.

IACUC guidelines are followed. The animals are given food and water ad libitum and are housed in an area separate from surgical rooms. Animals are anesthetized for surgery using an injection of ketamine (80-90 mg/kg) combined with xylazine (10-15 mg/kg). Sterile technique is used and the surgery is performed in a class 100 ISO Class 5 hood. The DBM implants are mildly hemostatic so there is no problem with continued bleeding of the defect. Animals are returned to their cages to recover and maintained on a heating pad until full alertness and recovery of mobility are observed.

Animals are routinely monitored daily and are observed for illness and distress by a veterinary surgeon every working day until sacrifice. If animals display any signs of illness, infection, or pain the veterinarian is consulted and euthanized if recommended. Animals are sacrificed by placing them in a hermetically-sealed box that is then flooded with carbon dioxide gas until the animals are unconscious. Following diaphragmatic puncture of the left and right hemithorax the implants are removed through an incision for further analyses.

### EXAMPLE 2

### mRNA Knockdown Using Double Stranded RNA

Double stranded siRNA containing sequences complementary to target mRNAs are resuspended in nuclease-free dI water to yield a 20 uM solution. For transfection of established cell lines siRNA is added to medium (DMEM lacking antibiotics and fetal bovine serum) to yield a final concentration of approximately 5 nM. Three volumes of HiPerFect siRNA Transfection Reagent (Qiagen) are added to the mixture, vortexed, and held for 10 minutes at room temperature prior to adding to cell monolayers. Except for refeeding cells with fresh, complete medium two days post-transfection, the cultures are left undisturbed at 37 C and 5% CO2.

At appropriate intervals following transfection total RNA is isolated from cell monolayers. Following removal of the medium the cells are rinsed with sterile PBS, pH 7.4 and then lysed in Trizol Reagent (Invitrogen). RNA is further purified by chloroform extraction followed by high-speed centrifugation and precipitated out of isopropanol. RNA is pelleted using a microfuge, the pellet is dried and then resuspended in RNAse-free water to yiled a final concentration of 0.5 ug/ul. One half microgram aliquots of RNA are used for Q-PCR reactions to compare relative levels of specific mRNAs among siRNA-treated and control samples. These assays are conducted using a SybrGreen PCR-Detection kit (Qiagen) in conjunction with a real-time PCR cycler. The accumulated amounts of target mRNA in control and experimental samples are normalized using GAPDH mRNA levels as a baseline and verification of knockdown is made using agarose gel electrophoresis of reaction products.

### EXAMPLE 3

### Runx2 Assays

Functional runx2 protein from experimental and control cell extracts is measured using a luciferase reporter gene assay. Into an indicator plasmid encoding a firefly luciferase gene under the control of an SV-40 promoter (Promega, Madison Wis.) was inserted six, tandemly repeated sequences from a runx2 binding element from the promoter of the osteocalcin gene (“6×OSE2”). This insertion renders the basal luciferase expression from the SV-40 promoter inducible to higher levels when active mm×2 protein is present. The amount of stimulation is proportional to the chemical amount of runx2 protein present and is a good indicator of the extent of osteoblastic determination displayed by a given cell type.

For these studies test cell lines are transfected with approximately 0.5 ug/ml plasmid using FuGene Transfection Medium (Roche) added directly to cell monolayers. Two days later the medium is removed, the cells are rinsed with PBS, pH 7.4 and the cells are lysed with 200 μl/6 well plate Luciferase Reporter Buffer (Promega). Twenty microliters of extract are combined with 200 ul Luciferase Substrate (Promega) and relative luminescence units are determined using a luminometer. Data are reported as RLU/ug protein in order to compare treatments.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope.

### REFERENCES CITED

Abe, E., Yammamoto, M., Taguchi, Y., Lecka-Czemik, B., O'Brien, C. A., Economide, A. N., Stahl, N., Jilka, R. L., and Manolagas, S. C. (2000). Essential requirement of bone morphogenetic proteins-2/4 for both osteoblast and osteoclast formation in murine bone marrow cultures from adult mice: antagonism by Noggin. J. Bone Mineral. Res. 15:663-675.
Bantounas, L. A., Phylactou, L. A. and Uney, J. B. (2004). RNA interference and the use of small interfering RNA to study gene function in mammalian systems. J. Molec. Endocrin. 33:545-557.
Benham, K., Brochmann, E. J. and Murray, S. S. (2004). Alkali-urea extraction of demineralized bone matrix removes noggin, an inhibitor of bone morphogenetic proteins. Connec. Tiss. Res. 45:257-260.
Bessho, K., Tagawa, T., and Murata, M. (1992). Comparison of bone matrix-derived bone morphogenetic proteins from various animals. J. Oral Maxillo. Surg. 50:496-501.
Bianco, P., Riminucci, M., Gronthos, S. and Robey, P. G. (2001). Bone marrow stromal cells: Nature, biology, and potential applications. Stem Cells 19:180-192.
Boden, S. D., Kang, J., Sandhu, H., and Heller, J. G. (2002). Use of recombinant human bone morphogenetic protein-2 to achieve posterolateral lumbar spine fusion in humans. Spine 27:2662-2673.
Canalis, E., Economides, A. N. and Gazzerro, E. (2003). Bone morphogenetic proteins, their antagonists and the skeleton. Endocrin. Rev. 24:218-235.
Chesmel, K. D., Branger, J., Wertheim, H. and Scarborough, N. (1998). Healing response to various forms of human demineralized bone matrix in athymic rat cranial defects. J. Oral Maxillo. Surg. 56:857-863.
Daluiski, A., Engstrand, T., Bahamonde, M. E., Gamer, L. W., Agius, E., Stevenson, S. L., Cox, K., Rosen, V., and Lyons, K. M. (2001). Bone morphogenetic protein-3 is a negative regulator of bone density. Nature Genet. 27:84-88.
Ducy, P. and Karsenty, G. (1995). Two distinct osteoblast-specific cis-acting elements control expression of a mouse osteocalcin gene. Molec. Cell. Biol. 15:1858-1869.
Ducy, P., Zhang, R., Geoffroy, V., Ridall, A. L., and Karsenty, G. (1997). Osf2/Cbfa1: A transcriptional activator of osteoblast differentiation. Cell 89:747-754.
Ducy, P., Starbuck, M., Priemel, M., Shen, J., Pinero, G., Geoffroy, V., Amling, M. and Karsenty, G. (1999). A Cbfa1-dependent genetic pathway controls bone formation beyond embryonic development. Genes Develop. 13:1025-1036.
Edwards, J. T., Diegmann, M. H., and Scarborough, N. L. (2003). Osteoinduction of human demineralized bone. Clin. Orthop. 357:219-228.
Einhorn, T. A. (1998). The cell and molecular biology of fracture healing. Clin. Orthop. Rel. Res. 355S:S7-S21.
Elbashir, S. M., Harborth, J., Lendeckel, W., Yalcin, A., Weber, K., and Tuschl, T. (2001). Duplexes of 21-nucleotide RNAs mediate RNA intereference in cultured mammalian cells. Nature 411:494-498.
Fujita, T., Izumo, N., Fukuyama, R., Meguro, T., Nakamuta, H., Kohno., T. and Koida, M. (2001). Phosphate provides an extracellular signal that drives nuclear export of Runx2/Cbfa1 in bone cells. Biochem. Biophys. Res. Comm. 280:348-352.
Gazzerro, E., Gangji, V. and Canalis, E. (1998). Bone morphogenetic proteins induce the expression of noggin which limits their activity in cultured rat osteoblasts. J. Clinical Invest. 102:2106-2114.
Geoffroy, V., Kneissel, M., Fournier, B., Boyde, A., and Matthias, P. (2002). High bone resorption in adult, aging transgenic mice overexpressing Cbfa1/Runx2 in cells of the osteoblastic lineage. Mol. Cell. Biol. 22:6222-6233.
Gerstenfeld, L. C., Cullinane, D. M., Barnes, G. L., Graves, D. T., and Einhorn, T. A. (2003). Fracture healing as a post-natal developmental process: Molecular, spatial, and temporal aspects of its regulation. J. Cell. Biochem. 88:873-884.
Groenvel, E. H. J. and Berger, E. H. (2000). Bone morphogenetic proteins in human bone regeneration. Eur. J. Endocrin. 142:9-21.
Gou, D., Jin, N. and Liu, L. (2003). Gene silencing in mammalian cells by PCR-based short hairpin RNA. FEBS Letts. 548:113-118.
Hartman, E. H. M., Pikkemaat, J. A., VanAsten, J. J., Vehof, J. W. M., Heerschap, A., Oyen, W. J. G., Spauwen, P. H. M. and Jansen, J. A. (2004). Demineralized bone matrix-induced ectopic bone formation in rats: In vivo study with follow-up by magnetic resonance imaging, magenetic resonance angiography, and dual-energy X-ray absorptiometry. Tiss. Engin. 10:747-754.
Hirata, K., Tsukazaki, T., Kadowaki, A., Furukawa, K., Shibata, Y., Moriishi, T., Okubu, Y., Bessho, K., Komori, T., Mizuno, A. and Yamaguchi, A. (2003). Transplantation of skin fibroblasts expressing BMP-2 promotes bone repair more effectively than those expressing Runx2. Bone 32:502-512.
Hsu, D. R., Economides, A. N., Wang, X., Eimon, P. M. and Harland, R. M. (1998). The Xenopus dorsalizing factor gremlin identifies a novel family of secreted proteins that antagonize BMP activities. Mol. Cell. 1:673-683.
Ichim, T. E., Li, M., Qian, H., Popov, I. A., Rycerz, K., Zheng, X., White, D., Zhong, R., Min, W.-P. (2004). RNA interference: A potent tool for gene-specific therapeutics. Am. J. Transplant. 4:1227-1236.
Jorgensen, N. R., Henriksen, Z., Sorensen, O. H. and Civitelli, R. (2004). Dexamethasone, BMP-2, and 1,25-dihydroxyvitamin D enhance a more differentiated osteoblast phenotype: Validation of an in vitro model for human bone marrow-derived primary osteoblasts. Steroids 69:219-226.
Karsenty, G., Ducy, P., Starbuck, M., Priemel, M., Shen, J., Geoffroy, V. and Amling, M. (1999). Cbfa1 as a regulator of osteoblast differentiation and function. Bone 25:107-108.
Katagiri, T., Yamaguchi, A., Komaki, M., Abe, E., Takahashi, N., Ikeda, T., Rosen, V., Wozney, J. M., Fujisawa-Sehara, A., and Suda, T. (1994). Bone morphogenetic protein-2 converts the differentiation pathway of C2Cl2 myoblasts into the osteoblast lineage. J. Cell Biol. 127:1755-1766.
Katagiri, T. and Takahashi, N. (2002). Regulatory mechanisms of osteoblast and osteoclast differentiation. Oral Dis.:147-159.
Kato, C., Kojima, T., Komaki, M., Mimori, K., Duarte, W. R., Takenaga, K., and Ishikawa, I. (2005). S100A4 inhibition by RNAi up-regulates osteoblast related genes in periodontal ligament cells. Biochem. Biophys. Res. Comm. 326:147-153.
Kim, D.-H., Behlke, M. A., Rose, S. D., Chang, M.-S., Choi, S. and Rossi, J. J. (2004). Synthetic dsDicer substrates enhance RNAi potency and efficacy. Nature Biotech. 23:222-226.
Kim, S., Koga, T., Isobe, M., Kern, B. E., Yokochi, T., Chin, Y. E., Karsenty, G., Taniguchi, T., and Takayanagi, H. (2003). Stat1 functions as a cytoplasmic attenuator of Runx2 in the transcriptional program of osteoblast differentiation. Genes Develop. 17:1979-1991.
Komori, T. (2000). A fundamental transcription factor for bone and cartilage. Biochem. Biophys. Res. Comm. 276:813-816.
Lee, M. H., Kim, Y.-J., Kim, H.-J., Park, H.-D., Kang, A.-R., Kyung, H.-M., Sung, J.-H., Wozney, J. M., Kim, H.-J., and Ryoo, H.-M. (2003). BMP-2-induced runx2 expression is mediated by Dlx5, and TGF-beta1 opposes the BMP-2-induced osteoblast differentiation by suppression of Dlx5 expression. J. Biol. Chem. 278:34387-34394.
Lee, S. B., Kim, Y. H., Chong, M. S., and Lee, Y. M. (2004). Preparation and characteristics of hybrid scaffolds composed of beta-chitin and collagen. Biomat. 25:2309-2317.
Lewis, D. L., Hagstrom, J. E., Loomis, A. G., Wolff, J. A. and Herweijer, H. (2002). Efficient delivery of siRNA for inhibition of gene expression in postnatal mice. Nature Genet. 32:107-108.
Li, X., Cui, Q., Kao, C., Wang, G. J. and Balian, G. (2003). Lovastatin inhibits adipogenic and stimulates osteogenic differentiation by suppressing PPAR gamma2 and increasing Cbfa1/Runx2 expression in bone marrow mesenchymal cell cultures. Bone 33:652-659.
Li, L., Xin, H., Huang, M., Zhang, X., Chen, Y., Zhang, S., Fu, X.-Y. and Chang, Z. (2004). CHIP mediates degradation of Smad proteins and potentially regulates Smad-induced transcription. Molec. Cell. Biol. 24:856-864.
Liao, S. S., Guan, K., Cui, F. Z., Shi, S. S. and Sun, T. S. (2003). Lumbar spinal fusion with a mineralized collagen matrix and rhBMP-2 in a rabbit model. Spine 28:1954-1960.
Marazzi, G., Wang, Y. and Sassoon, D. (1997). Msx2 is a transcriptional regulator in the BMP4-mediated programmed cell death pathway. Dev. 4Biol. 186:127-138.
McManus, M. T. and Sharp, P. A. (2002). Gene silencing in mammals by small interfering RNAs. Nature Rev. 3:737-747.
Minakuchi, Y., Takeshita, F., Kosaka, N., Sasaki, H., Yamamoto, Y., Kouno, M., Honma, K., Nagahara, S., Hanai, K., Sano, A., Kato, T., Terada, M. and Ochiya, T. (2004). Atelocollagen-mediated synthetic small interfering RNA delivery for effective gene silencing in vitro and in vivo. Nucl. Acids Res. 32:1-7.
Miyazono, K. (1999). Signal transduction by bone morphogenetic protein receptors: functional roles of SMAD proteins. Bone 25:91-93.
Mushik, M., Schlenzka, Ritsila, V., D., Tennstedt, C., and Lewandrowski, K. U. Experimental anterior spine fusion using bovine bone morphogenetic protein: A study in rabbits. J. Orthop. Sci. 5:165-170.
Nobta, M., Tsukazaki, T., Shibata, Y., Xin, C., Monishi, T., Sakano, S., Shindo, H. and Yamaguchi, A. (2005). Critical regulation of bone morphogenetic protein induced osteoblastic regulation by Delta?Jagged1-activated Notch1 signaling. J. Biol. Chem. 280:15842-15848.
Ochiya, T., Takahama, Y., Nagahara, S., Sumita, Y., Hisada, A., Itoh, H., Nagai, Y., and Terada, M. (1999). New delivery system for plasmid DNA in vivo using atelocollagen as a carrier material: The minipellet. Nature Med. 5:707-710.
Onichtchouk, D., Chen, Y.-G., Dosch, R., Gawntka, V., Delius, H., Massague, J., and Niehrs, J. (1999). Silencing of TGF-beta signaling by the pseudoreceptor BAMBI. Nature 401:480-485.
Piccolo, S., Sasai, Y., Lu, B., and DeRobertis, E. M. (1996). Dorsoventral patterning in Xenopus: inhibition of ventral signals by direct binding of chordin to BMP4. Cell 86:589-598.
Piccolo, S., Agius, E., Leyns, L., Bhattarcharyya, S., Grunz, H., Bouwmeester, T., and DeRobertis. E. M. (1999). The head inducer Cerebus is a multifunctional antagonist of Nodal, BMP, and Wnt signals. Nature 397:707-710.
Prince, M., Banerjee, C., Javed, A., Green, J., Lian, J. B., Stein, G. S., Bodine, P. V. N. and Komm, B. S. (2001). Expression and regulation of Runx2/Cbfa1 and osteoblast phenotypic markers during the growth and differentiation of human osteoblasts. J. Cell. Biochem. 80:424-440.
Ross, J. J., Shimmi, O., Vilmos, P., Petryk, A., Kim, H., Gaudenz, K., Hermanson, S., Ekker, S. C., O'Connor, M. B., and Marsh, J. L. (2001). Twisted gastrulation is a conserved extracellular BMP antagonist. Nature 410:487-492.
Sakuta, H., Suzuki, R., Takahashi, H., Kato, A., Shintani, T., Iemura, S., Yamamoto, T., Ueno, N. and Noda, M. (2001). Ventropin: a BMP4 antagonist expressed in a double gradient pattern in the retina. Science 293:111-115.
Sampath, T. K., Muthukumaran, N. and Reddi, A. H. (1987). Isolation of osteogenin, an extracellular matrix-associated, bone inductive protein, by heparin affinity chromatography. Proc. Natl. Acad. Sci. USA 84:7109-87113.
Sandhu, H. S., Khan, S. N., Suh, D. Y. and Boden, S. D. (2001). Demineralized bone matrix, bone morphogenetic proteins and animal models of spine fusion: An overview. Eur. Spine J. 10: S122-S131.
Schroeder, T. M., Kahler, R. A., Li, X. and Westendorf, J. J. (2004). Histone deacetylase-3 interacts with Runx2 to repress the osteocalcin promoter and regulate osteoblast differentiation. J. Biol. Chem. 279:41998-42007.
Shankar, P., Manjunath, N., and Lieberman, J. (2005). The prospect of silencing disease using RNA interference. JAMA 293:1367-1373.
Silva, J. M., Mizuno, H., Brady, A., Lucito, R. and Hannon, G. J. (2004). RNA interference microarrays: High-throughput loss-of-function genetics in mammalian cells. Proc. Natl. Acad. Sci. 101:6548-6552.
Song, E., Zhu, P., Lee, S.-K., Chowdury, D., Kussman, S., Dykxhoom, D. M., Feng, Y., Palliser, D., Weiner, D. B., Shankar, P., Marasco, W. A. and Lieberman, J. (2005). Antibody mediated in vivo delivery of small interfering RNAs via cell-surface receptors. Nature Biotech. 23:709-717.
Soutschek, J., Akinc, A., Bramlage, B., Charisse, K., Constein, R., Donoghue, M., Elbashir, S., Gelck, A., Hadwiger, P., Harborth, J., John, M., Kesavan, V., Lanine, G., Pandey, R. K., Racie, T., Rjeev, K. G., Rohl, I., Toudjarska, I., Wang, G., Wuschko, S., Bumcrot, D., Koteliansky, V., Limmer, S., Manoharan, M., Vornlocher, H.-P. (2004). Therapeutic silencing of an endogenous gene by systemic administration of modified siRNAs. Nature 432:173-178.
Stanley, E., Biben, C., Kotecha, S., Fabri, L., Tajbakhsh, S., Wang, C.-C., Hatzistavrou, T., Roberts, B., Drinkwater, C., Lah, M., Buckingham, M., Hilton, D., Nash, A., Mohun. T., and Harvey, R. P. (1998). DAN is a secreted glycoprotein related to Xenopus cerebus. Mech. Develop. 77:173-184.
Stein, G. S., Lian, J. B., vanWijnen, A. J., Stein, J. L., Montecino, M., Javed, A., Zaidi, S. K., Young, D. W., Choi, J.-Y., and Pockwinse, S. M. (2004). Runx2 control of organization, assembly and activity of the regulatory machinery for skeletal gene expression. Oncogene 23:4315-4329.
Takei, Y., Kadomatsu, K., Yuzawa, Y., Matsuo, S., and Muramatsu, T. (2004). A small interfering RNA targeting vascular endothelial growth factor as cancer therapeutics. Cancer Res. 64:3365-3370.
Urist; Marshall R. U.S. Pat. Nos. 4,857,456; 4,795,804; 4,789,732; 4,761,471; 4,619,989; 4,596,574; 4,563,489; 4,526,909; 4,455,256; 4,294,753.
vanBezooijen, R. L., Karperien, M., Visser, A., Hamersma, H., Winkler, D., Hayes, T., Skonier, J., Staehling-Hampton, K., Latham, J. A., Papapoul;os, S. E., and Lowik, C. W. G. (2002). BMP-antagonist sclerostin is expressed in mineralized bone and blocks BMP-induced bone formation. J, Bone Mineral Research 16 (Suppl. 1):S163.
Viereck, V., Siggelkow, H., Tauber, S., Raddatz, D., Schutze, N., and Hufner, M. (2002). Differential regulation of Cbfa1/Runx2 and osteocalcin gene expression by vitamin-D3, dexamethasone and local growth factors in primary human osteoblasts. J. Cell. Biochem. 86:848-856.
Wang, J. and Glimcher, M. J. (1999). Characterization of matrix-induced osteogenesis in rat calvarial defects: I. Differences in the cellular response to demineralized bone matrix implanted in calvarial defects and in subcutaneous sites. Calcif. Tiss. Inter. 65:156-165.
Xiao, L., Naganawa, T., Obugunde, E., Gronowicz, G., Ornitz, D. M., Coffin, J. D. and Hurley, M. M. (2004). Stat1 controls postnatal bone formation by regulating fibroblast growth factor signaling in osteoblasts. J. Biol. Chem. 279:27743-27752.
Yang, S., Wei, D., Wang, D., Phimphilai, M., Krebsbach, P. H., and Franceschi, R. T. (2003). In vitro and in vivo synergistic interactions between the Runx2/Cbfa1 transcription factor and bone morphogenetic protein-2 in stimulating osteoblast differentiation. J. Bone Mineral Res. 18:705-715.
Ying, S.-X., Hussain, Z. J. and Zhang, Y. E. (2003). Smurf1 facilitates myogenic differentiation and antagonizes the bone morphogenetic protein-2-induced osteoblast conversion by targeting Smad5 for degradation. J. Biol. Chem. 278:39029-39036.
Yoshida, Y., Tanaka, S., Umemori, H., Minowa, O., Usul, M., Ikematsu, N., Hosoda, E., Imamura, T., Kuno, J., Yamashita, T., Miyazono, K., Noda, M., Noda, T. and Yamamoto, T. (2000). Negative regulation of BMP/Smad signaling by Tob in osteoblasts. Cell 103:1085-1097.
Yoshikawa, H., Tanaguchi, S.-i., Yamamura, H., Mori, S., Sugimoto, M., Miyado, K., Nakamura, K., Nakao, K., Katsuki, M., Shibata, N., and Takahashi, K. (1998). Mice lacking smooth muscle calponin display increased bone formation that is associated with the enhancement of bone morphogenetic responses. Genes to Cells 3:685-695.
Yoshimura, Y., Nomura, S., Kawasaki, S., Tsutsumimoto, T., Shimizu, Y. and Takaoka, K. (2001). Co-localization of Noggin and BMP4 during fracture healing. J. Bone Mineral. Res. 16:876-884.
Zhang, Y.-W., Bae, S.-C., Huang, G., Fu, Y.-X., Lu, J., Ahn, M.-Y., Kanno, Y., Kanno, T., and Ito, Y. (1997). A novel transcript encoding an N-terminally truncated AML1/PEBP2 alpha/beta protein interferes with transactivation and blocks granulocyte differentiation of 32Dc13 myeloid cells. Mol. Cell. Biol. 7:4133-4145.
Zhang, Y., Chang, C., Gehling, D. J., Hemmati-Brivanlou, A., and Derynck, R. (2001). Regulation of SMAD degradation and activity by SMURF2, and E3 ubiquitin ligase. Proc. Natl. Acad. Sci. USA 98:974-979.
Zhao, M., Qiao, M., Harris, S. E., Oyajobi, B. O., Mundy, G. R. and Chen, D. (2004). Smurf1 inhibits osteoblast differentiation and bone formation in vitro and in vivo. J. Biol. Chem. 279:12854-12859.
Zimmerman, L. B., DeJesus-Escobar, J. M., and Harland, R. M. (1996). The Spemann organizer signal Noggin binds and inactivates bone morphogenetic protein-4. Cell 86:599-606.
Zuk, P. A., Zhu, M., Mizuno, H., Huang, J., Futrell, J. W., Katz, A. J., Benhaim, P., Lorenz, H. P. and Hedrick, M. H. (2001). Multilineage cells from human adipose tissue: Implications for cell-based therapies. Tiss. Engin. 7:211-228.

What is claimed is:

1. An siRNA molecule comprising a double stranded RNA portion having about 5 to 40 bp said molecule down-regulating the expression in a mammal of a protein inhibitor of osteogenesis or chondrogenesis.

2. The siRNA molecule of claim 1, wherein said mammal is human.

3. The siRNA molecule of claim 2, wherein the double stranded RNA portion consists of 13 to 30 bp.

4. The siRNA molecule of claim 3, wherein the double stranded RNA portion consists of 19 to 23 bp.

5. The siRNA molecule of claim 3, wherein said protein inhibitor is a protein inhibitor is selected from the group consisting of HDAC-3, STAT-1, SMURF-1, SMURF-3, SMURF-8, Tob, Calponin, and noggin.

6. The siRNA molecule of claim 5, wherein said protein inhibitor is HDAC-3 or SMURF-1.

7. The siRNA molecule of claim 6, wherein said protein inhibitor is HDAC-3.

8. The siRNA molecule of claim 7, wherein the double stranded RNA portion is selected from the group consisting of the following combinations of sense and anti-sense strands: SEQ ID NO: 9/SEQ ID NO: 10; SEQ ID NO: 11/SEQ ID NO: 12; SEQ ID NO: 13/SEQ ID NO: 14; and SEQ ID NO: 15/SEQ ID NO: 16.

9. The siRNA molecule of claim 7, wherein the double stranded RNA portion comprises:
a first RNA strand selected from the group consisting SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16;
and a second RNA strand that is a complement of the first RNA strand.

10. The siRNA molecule of claim 6, wherein said protein inhibitor is SMURF-1.

11. The siRNA molecule of claim 10, wherein the double stranded RNA portion is selected from the group consisting of the following combinations of sense and anti-sense strands: SEQ ID NO: 1/SEQ ID NO: 2; SEQ ID NO: 3/SEQ ID NO: 4; SEQ ID NO: 5/SEQ ID NO: 6; and SEQ ID NO: 7/SEQ ID NO: 8.

12. The siRNA molecule of claim 10, wherein the double stranded RNA portion comprises:
a first RNA strand selected from the group consisting SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8;
and a second RNA strand that is a complement of the first RNA strand.

13. An siRNA molecule comprising a double stranded RNA portion having about 5 to 40 bp said molecule down-regulating the expression in a mammal of a protein inhibitor of osteogenesis or chondrogenesis.

14. An siRNA molecule comprising a double stranded RNA portion and two single stranded DNA portions, the double stranded RNA portion having about 15-40 bp, the two single stranded DNA portions may be the same or different, each DNA portion comprising two deoxynucleotides which may be the same or different, each DNA portion being attached to the 3′ end of each RNA strand, the siRNA molecule down-regulating the expression in a mammal of a protein inhibitor of osteogenesis.

15. The siRNA molecule of claim 14 wherein the patient is human.

16. An implantable bone graft comprising in combination a sterilized segment of allograft or xenograft bone in combination with an siRNA molecule of claim 1.

17. An implantable bone graft comprising in combination a sterilized segment of allograft or xenograft bone in combination with an siRNA molecule of claim 9.

18. An implantable bone graft comprising in combination a sterilized segment of allograft or xenograft bone in combination with an siRNA molecule of claim 1.

19. A method of enhancing osteogenesis in a mammalian patient at the site of an osteo implant comprising
a. providing an osteo implant to a site in a mammalian patient in need of an osteo implant;
b. providing to the patient at the site of the implant an siRNA molecule of claim 14 whereby an inhibitor to osteoblastic activity is down-regulated.

20. An implantable composition for use in a mammal comprising in combination a demineralized bone matrix and an siRNA molecule of claim 1.
